(19) United States
(12) Patent Application Publication
(10) Pub. No.: US 2016/0166180 A1
(43) Pub. Date: Jun. 16, 2016

(54) Enhanced Real Time Frailty Assessment for Mobile

(71) Applicant: David Martin, San Francisco, CA (US)

(72) Inventor: David Martin, San Francisco, CA (US)

(73) Assignee: David Martin, San Francisco, CA (US)

(21) Appl. No.: 14/932,591

(22) Filed: Nov. 4, 2015

Related U.S. Application Data
(60) Provisional application No. 62/090,698, filed on Dec. 11, 2014.

(51) Int. Cl. A61B 5/11; A61B 5/00; G01P 15/00

(52) U.S. Cl. CPC A61B 5/112; G01P 15/00; A61B 5/726; A61B 5/6803; A61B 5/6898; A61B 5/7207; A61B 5/725; A61B 5/1112; A61B 5/4866; A61B 5/7278; A61B 5/486; A61B 5/742; A61B 5/4023; A61B 2562/0219; A61B 2560/0223

(57) ABSTRACT

Some embodiments of the invention provide methods and apparatus for enhanced real time frailty assessment leveraging a mobile or wearable device. In some embodiments, the mobile or wearable device user's gait characteristics are determined in real time, and said information is integrated with additional information comprising the user's balance evaluation and contextual information obtained making use of the mobile or wearable device sensors, in order to deliver an enhanced frailty assessment.

### CROSS-REFERENCE TO RELATED APPLICATIONS

This application claims priority from U.S. Provisional Patent Application Ser. No. 62/090,698, by David Martin, filed on Dec. 11 2014, entitled “Enhanced Real Time Frailty Assessment for Mobile”.

### BACKGROUND

1. Field

This application relates to mobile and wearable devices, specifically to methodologies to leverage user's gait characteristics, balance and other factors to assess frailty.

2. Discussion of Related Art

State-of-the-art technologies and devices enable activity recognition with certain degree of accuracy, depending on the type and quality of the sensors and methodologies employed, among other factors. However, currently available approaches may suffer from important inaccuracies due to variations in movement attributes during the physical activity. Particularities in gait characteristics and other conditions may add to the difficulties that some approaches face to accurately monitor activities. There is a need to efficiently leverage the sensors embedded in mobile and wearable devices to precisely determine gait characteristics, assess balance and determine contextual information, and together with other factors, enable accurate frailty assessment.

### BRIEF DESCRIPTION OF THE DRAWINGS

FIG. 1A represents an example of mobile device user walking with the device.

FIG. 1B represents an example of wearable device user running with the device.

FIG. 2 represents an example of mobile and/or wearable device users performing some gait activity with their devices in a networking environment.

FIG. 3 shows an example of an embodiment of the presentation of contextual information on a mobile and/or wearable device.

FIG. 4 shows an example of another embodiment of the presentation of contextual information on a mobile and/or wearable device.

FIG. 5 displays a schematic frailty assessment model for one embodiment.

FIG. 6 illustrates a process flow diagram for the user's dynamics information determination according to one embodiment.

FIG. 7 illustrates a flow diagram for the process to enhance a user's dynamics and localization information according to one embodiment.

### DETAILED DESCRIPTION OF EMBODIMENTS OF THE INVENTION

Although the following text sets forth a detailed description of numerous different embodiments, it should be understood that the legal scope of the description is defined by the words of the claims set forth at the end of this disclosure. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

It should also be understood that, unless a term is expressly defined in this patent using the sentence “As used herein, the term‘ ’is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning.

Some inventive functionality and inventive principles may be implemented with or in software programs or instructions and integrated circuits (ICs) such as application specific ICs. In the interest of brevity and minimization of any risk of obscuring the principles and concepts according to the present invention, discussion of such software and ICs, if any, is limited to the essentials with respect to the principles and concepts within some of the embodiments.

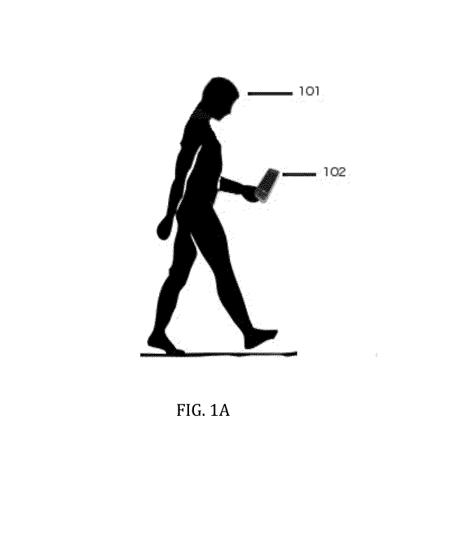

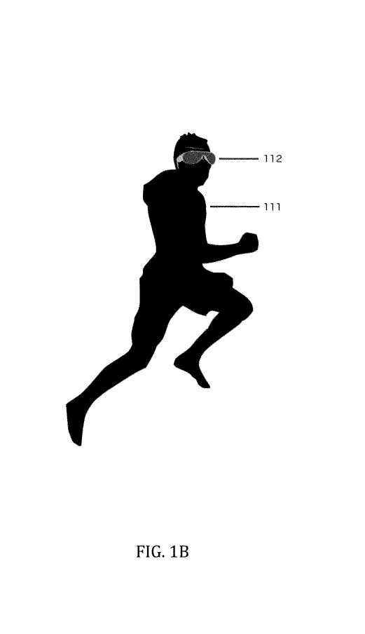

FIG. 1A represents an individual, (101), walking with a mobile device, (102). In some embodiments, individual (101) may be performing any kind of walking, jogging, running, sprinting, or any other type of gait activity. In other embodiments, individual (101) may be performing any kind of activity. In some embodiments, (101) could be a human, a robot, or a non-human animal, while (102) could be any type of mobile, wearable or any other type of device (capable of being positioned in any part of the body or any where), with any combinations thereof also possible. By way of example, and not limitation, (102) may represent a smartphone held in the hand or hands while individual (101) walks looking at its screen. In some embodiments, device (102) could be positioned in any pocket of individual (101), or held in any hand while walking without facing the individual (by way of example, and not limitation, when the individual does not look at the device screen), or placed in any type of clothing or any kind of bag or accessory brought by the individual. In some embodiments, device (102) could be positioned, placed or in any way attached to any part of the individual's body or accessories. By way of example, and not limitation, in some embodiments (102) may represent any type of hands-free device, virtual reality device, eyewear or glasses that individual (101) is wearing in any way attached or positioned on his/her face, head, or any other place of his/her body or accessories. In this sense, FIG. 1B represents an example of one embodiment in which individual (111) is running while wearing a device in the form of glasses (112). In some embodiments (112) may represent any type of virtual reality device, eyewear, glasses or any other type of wearable or mobile device that individual (111) is wearing in any way attached or positioned on his/her face, head, or any other place of his/her body or clothes or accessories, and individual (111) may be performing any kind of walking, jogging, running, sprinting, or any other type of gait activity. In other embodiments, individual (111) could be performing any kind of activity. In general, any examples/embodiments applicable to (101) and (102), are applicable to (111) and (112) respectively, and vice-versa, with any variations and/or combinations also possible.

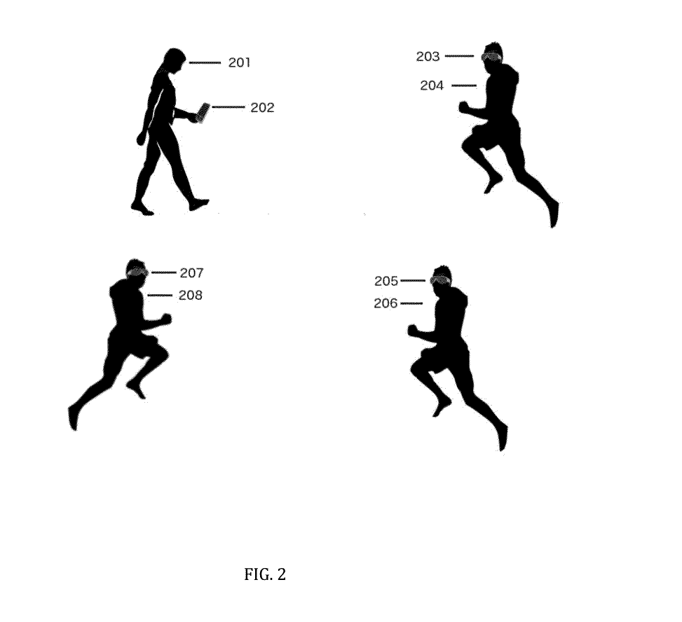

FIG. 2 represents an example of an embodiment in which four individuals (201), (204), (206), (208) participate in a networking environment; in this particular embodiment, each individual has one device: individual (201) is walking and has device (202), which may represent a smartphone, phablet, tablet, or any other type of device, including by way of example without limitation, any of the types of devices that (112) and/or (102) may represent. Individual (204) is running and has device (203), which may represent any type of electronic glasses, a virtual reality device, or any kind of wearable device worn on any part of the body, or any other type of device, including by way of example without limitation, any of the types of devices that (112) and/or (102) may represent. In a similar way, individuals (206) and (208) are running and wearing their own devices (205) and (207) respectively, which again could be any of the types of devices that (112) and/or (102) may represent.

In other embodiments, the number of individuals participating in a networking environment may be any, each one of the individuals may have any number of devices of any type positioned/attached/located/worn on any place, and each one of the individuals may perform any type of walking, jogging, running, sprinting, or any other type of activity regardless of the type of device and/or their position. In some embodiments, the individuals (201), (204), (206), (208) and/or any other number of individuals participating in a networking environment, may all be physically located next to each other in approximately the same location. In other embodiments, one or more (or all) individuals may be physically located in different and/or distant locations. By way of example without limitation, each individual could be in a different city.

In some embodiments, communication between devices (202), (203), (207), (205) and/or others to enable the networking environment may leverage any means, including, by way of example without limitation, any wireless and/or any other type of communications technology, such as LTE, UMTS, GSM, WiFi, Bluetooth and/or any other kind and combinations thereof. In some embodiments, the means of communications and/or their properties may be varied at any time and depending on any reason. These changes and/or choices may depend on a plurality of factors, including, by way of example without limitation, network availability, physical proximity of individuals, power management, communication efficiency, specific usage plans, etc. In some embodiments where the devices are in close proximity, they may communicate directly with each other using any kind of short-range communications technology or any other type of means and/or technology without the need to relay on other communications networks elements such as cellular base stations or WiFi access points.

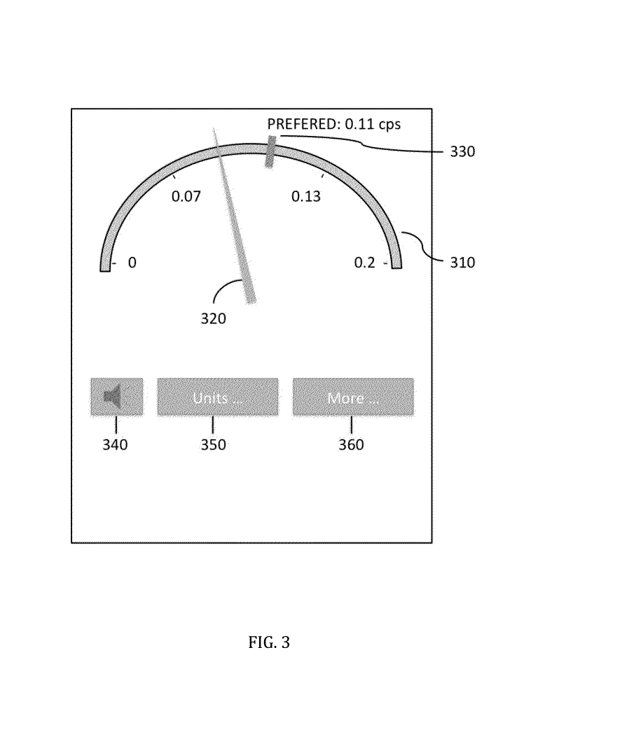

In some embodiments, any contextual information may be displayed directly on the user's device display. By way of example and not limitation, the velocity of the user may be displayed in real time (typically, fractions of a second) on the mobile device display; in other embodiments, any other type of information (related or not to the user's activity) may be displayed in real time on the mobile device. In some embodiments, information directly related to the user's gait velocity (e.g. calories burned per time unit) may be displayed on the mobile device, as shown in FIG. 3, which illustrates an example of the many possible embodiments. In this sense, it is interesting to note that the relationship between velocity and calories burned has been extensively studied (e.g. “Medicine and Science in Sports and Exercise”, 2011, by Ainsworth B E, Haskell W L, Herrmann S D, Meckes N, Bassett Jr D R, Tudor-Locke C, Greer J L, Vezina J, Whitt-Glover M C, Leon A S), and said relationship (e.g. Calories per second equal to the user Weight (in Kg) multiplied by MET/3600, where MET is well known (e.g. MET for walking equals 1 plus velocity in mph times 0.7663 if velocity lower than 3.5, or −6.69 plus velocity in mph times 2,642 otherwise)) may be leveraged in some embodiments in order to determine (and display) the calories per time unit burned by a mobile device user; in some embodiments, any other methodologies, approaches, technologies, devices, sensors, and/or modifications, and/or variations, and/or combinations thereof may be leveraged to determine (and display) the calories per time unit burned (or any other related or not magnitude, indication, variable and/or combinations thereof) by a mobile device user.

Some embodiments may present the real time value and evolution of the contextual information on the mobile device. Other embodiments may display the contextual information on an external managing or monitoring entity, which may comprise computing and storing resources. Other embodiments with different configurations and/or combinations thereof are also possible. In some embodiments, a semicircular scale may be used to represent the calories burned per time unit (310), and it may be calibrated in different and adaptable units and values depending on context. By way of example and not limitation, calories burned may be represented from 0 calories-per-second (cps) to 0.2 cps. In some embodiments, different types and/or number of indications, scales, units, values, etc. and combinations thereof may be used and/or displayed. By way of example without limitation, the calories (or Kcal or any other magnitude) burned may be represented in terms of minutes (or hours or any other magnitude) rather than seconds, or any other possibilities and combinations thereof. In some embodiments, cumulative values (e.g. total calories or Kcals burned) may be used and/or displayed, with any possibilities in terms of time origin (e.g. total calories burned for the last hour, or since 25 minutes ago, etc.). Some embodiments may use and/or display any other possible indication, variable, magnitude, and/or variations and/or combinations thereof. In addition, the scale may include a variety of features, such as the optimum or preferred calories burning rate (330) or any others, using and/or displaying any type of units, characteristics, parameters and/or combinations thereof. In some embodiments, the device user may select the type of units, ranges, features, etc. to be displayed, leveraging configuration buttons included in the display screen, or through a settings screen displayable through a variety of methods, or through any other option or methodology. In other embodiments, the mobile or wearable device itself will automatically adjust the units, ranges, features, etc. according to a variety of criteria, such as user's activity, location, profile, historic data, any contextual information, or any other possibility. In other embodiments, any other possibilities and/or options and/or criteria and/or combinations thereof may be used.

By way of example without limitation, the features may be average values or personalized values for each particular user, while other embodiments may use other types of features and/or combinations thereof, or other embodiments may use a semicircle with different colors representing calories burned.

In some embodiments, the representation of a moving needle (320) may be leveraged to indicate the real time value of the measured magnitude (e.g. calories burned per time unit). In other embodiments, other representations may be leveraged to indicate the real time value of the measured magnitude, including but not limited to, the surface of a varying semicircle whose angle grows from 0 degrees to 180 degrees depending on the magnitude value. In other embodiments, semi-arcs or other types of geometries, shapes, sizes, figures, etc. may also be leveraged. In some embodiments, combinations of geometries and/or color may also be leveraged to display the magnitude information. In some embodiments, the presentation of information to the user or to any type of managing or monitoring entity may be performed personalized and in any of several ways including, by way of example, and not limitation, visual, acoustic, etc. For example, a button for sound (340) may be used to enable or disable the acoustic delivery of contextual information. This button may also be leveraged to enable or disable playing music or other encouraging sound in the background, or to trigger an out-loud-reader mechanism to read-out-loud contents on the display (e.g. text from a website, messages received from friends, etc.) when predetermined and/or selectable thresholds or levels on the measured magnitude or general context are reached. Another button may be used to change the units of the measured magnitude (350), for example, calories per second, kilocalories per hour, etc. In some embodiments, automatic localization or other means may be leveraged to infer the country of the user and automatically adapt units, language, and other variables. Additional buttons (360) may also be employed for other purposes, including but not limited to, displaying a time evolution of the measured magnitude, dynamics, or general context over a selected or available period of time, allow personalized calibration, set preferences, etc.

In some embodiments, any element(s) described for any figure or embodiment may be optional, or any of them and any other additional element(s) with any features and/or combinations thereof, may also be included in any fashion in any figure or embodiment.

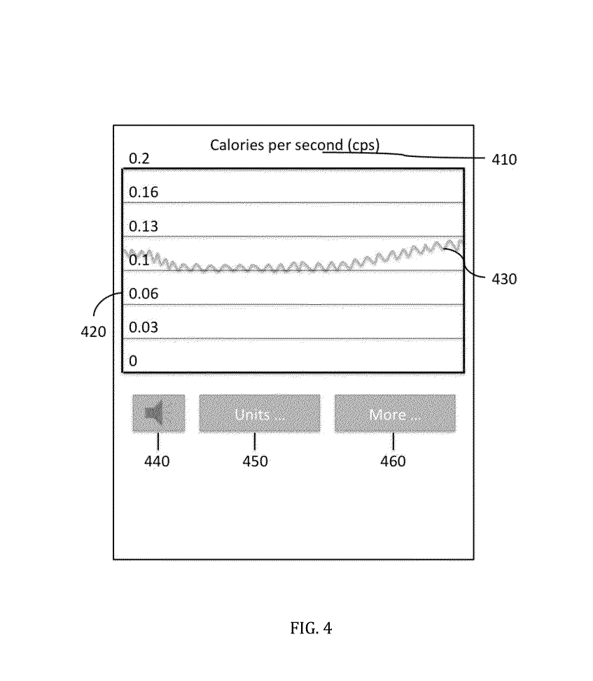

FIG. 4 represents an embodiment of a representation of the user's calories burned per time unit; in other embodiments, any other information and/or gait characteristic or attribute (e.g. stride length, cadence, total or cumulative calories burned, etc. and/or variations and/or combinations thereof) or related information may be represented. In a particular embodiment, real-time feedback and/or comparisons with other users and/or devices may also be allowed in any fashion.

In some embodiments, element (410) is an optional label and may provide the information on the magnitude being displayed together with any form of any possible units in which it may be measured (if applicable), or any other type of information of any nature, including abbreviations; particular examples may include: calories per second (cps), calories per minute, kilocalories per hour, and/or any other units and/or their abbreviations, and/or variations and/or combinations thereof. In other embodiments, if this label is included, depending on a variety of circumstances/conditions/choices, it may present any type of statements (e.g. “Calories burned (cps)”, “Calories (cps)”, “Kcal burned”, etc.), forms, shapes, positions, nature (e.g. any picture, icon, multimedia element, etc. and combinations thereof), or any other property and/or combinations thereof. In some embodiments, element (420) is also optional and it represents chart axes or grid for clarity purposes (any of its components is also optional and may have any characteristic); in some embodiments, the vertical axis may be scaled in any way depending on the magnitude being displayed, and hold a set of representative figures together with any type of unit, statement, or any other element of any nature, form, or any other characteristic, and arranged/aligned/distributed in any way; in a particular embodiment, the vertical axis is scaled from 0 to 0.2 in consecutive numbers (representing units of calories burned per second), and horizontal lines may cross the chart for each one of the presented numbers.

In some embodiments, the scale may additionally include a variety of features, such as the preferred magnitude rate or others. These features may be average values or personalized values for each particular user. Other embodiments may use any other types of features and/or combinations thereof. In other embodiments, any or all of the horizontal bars and/or numbers along the vertical axis and/or any other element may be optional (e.g. it may not be displayed at any time for any reason) and if they are displayed, any or all of the referred elements or any other of any type that may also be added, may present any properties/features and combinations thereof.

Element (430) represents the measurement of the magnitude (e.g. calories burned per second) or any other related information being displayed. In a particular embodiment, it is a continuous line or curve (linking points ordered in time, each point corresponding to each measurement of the magnitude) freely following the measurements, having up to a predetermined threshold in the number of points, and accepting a new point to be displayed appended in a continuous form to the right edge of the curve every time a new measurement arrives. When the threshold in the number of points is reached, every time a new measurement arrives, the first point from the left edge (which in this embodiment represents the oldest measurement) is discarded, and the rest of points (except for the new one included at the right edge) are offset one position towards the left, thus giving the impression of a continuous flow of points following the arriving measurements. In some embodiments, the threshold in the maximum number of points in element (430) may be set to a fixed amount (e.g. a hundred or any other number), while in other embodiments it may be variable and depend on a variety of factors/circumstances/conditions, user's choices or any other reason. In some embodiment, any other type of indication and combinations thereof may be used instead of simple points or dots placed at the actual measurement value, such as, by way of example without limitation, any number of stars, squares, diamond shaped icons, any other type of polygon/icon/drawing/entity/element, any type of dotted lines/curves, any type of line/curve from any edge of the chart (or any other place) to the actual measurement value, any type of rectangle or any other polygon, icon, drawing, entity, element covering an area from any edge of the chart (or any other place) to the actual measurement value, or any other element(s) with any properties distributed and/or organized in any way, including any modifications and/or combinations thereof. In some embodiments, the indications may represent any type of information, including by way of example without limitation, the actual raw measurement of the magnitude being displayed, any value derived from the raw measurement or from any group of measurements (e.g. mean, standard deviation, etc.), or any other value, information, processed data or any other element in any way related with the magnitude and combinations thereof.

In some embodiments, the frequency at which a new point (or any indication of any type corresponding to measurements or any other data) is introduced in element (430) may be the frequency at which a new measurement is generated. In a particular embodiment presenting (by way of example) calories per second derived from the user's velocity, the use of (by way of example) methodology based on the application of the wavelet transform to the acceleration signal, would allow a new measurement every time a new acceleration value is available; consequently, the frequency at which a new measurement is generated may be equal to the accelerometer sampling frequency; in other words, the frequency at which the calories related magnitude is updated may be equal to the accelerometer sampling rate, which in some embodiments may be higher than the user's step frequency. In some embodiments, other frequencies (lower or higher) may also be possible making use of different techniques, including by way of example without limitation, the use of any extra device, hardware, software, up sampling, down sampling, filtering, or any other techniques, tools and/or methodologies and any variations and/or combinations thereof. By way of example without limitation, in some embodiments the update frequency for the calories related magnitude may be 60 Hz or 120 Hz depending on device hardware and other circumstances/conditions/choices, therefore achieving an enhanced real-time presentation of information (and user experience) in comparison with other methods with lower update rates; in some embodiments, when the user's step frequency is below 1 Hz (e.g. 0.5 Hz), the update rate may also be chosen just above the user's step frequency (e.g. 0.6 Hz), or above 1 Hz, or set as the accelerometer sampling rate (e.g. 60 Hz or 120 Hz) to enhance the real-time presentation of information (and user experience); other embodiments may choose any other update frequency or characteristic by modifying any settings, conditions, and/or choices of the referred and/or any other method. Other embodiments may employ any modification to any aspect previously mentioned, and/or combinations thereof.

In some embodiments, the presentation of information to the user or to any type of managing or monitoring entity may be performed personalized and in any of several ways including, by way of example, and not limitation, visual, acoustic, etc. For example, a button for sound (440) may be used to enable or disable the acoustic delivery of any type of data/information (including by way of example without limitation, any kind of multimedia streaming and combinations thereof). This button may also be leveraged to enable or disable playing music or other encouraging sound in the background, or to trigger an out-loud-reader mechanism to read-out-loud contents on the display (e.g. text from a website, messages received from friends, etc.) when predetermined and/or selectable thresholds or levels on the user's calories burned or general context are reached. Another button may be used to change the units of the magnitude being displayed (450), for example, calories burned per minute, kilocalories per hour, etc. In some embodiments, automatic localization or other means may be leveraged to infer the country of the user and automatically adapt units, language, and other variables. Additional buttons (460) may also be employed for other purposes, including but not limited to, displaying the magnitude in different format, or displaying different information, set preferences, modify any aspect or property of the presentation and/or any application, etc. and combinations thereof.

In a particular embodiment, the background of the display/screen in FIG. 4 (including the background of the chart (420)) may be set to a dark color (e.g. black) while the rest of elements (axes or grid of the chart (420), and elements (410), (430), (440), (450), (460)) are set to light colors. Any other settings, modifications, and combinations thereof are also possible. In some embodiments, any of the elements in FIG. 4 and/or any of their sub-elements and/or any additional elements not described herein may be optional (e.g. may or may not be displayed) and/or may be set and/or modified and/or organized in any other way, including combinations thereof, and/or any feature or any other property about any or all of them may be set or modified in any fashion, including combinations thereof.

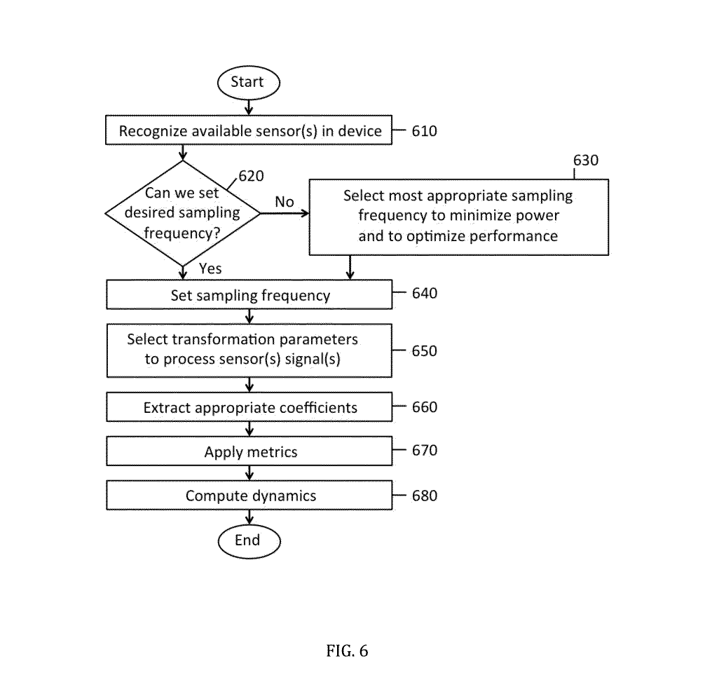

In some embodiments, only an accelerometer (tri-axial or any other type) embedded in the user's device may be used as sensor to determine the user's information, while other embodiments may employ additionally and/or independently any other type of sensor(s), device(s), sensor(s) embedded in other device(s), and/or any modifications and/or combinations thereof; by way of example without limitation, a tri-axial accelerometer in combination with GPS, or in combination with GPS and/or any other sensor (e.g. gyroscope, magnetometer, pressure sensor, etc.), or GPS on its own, or accelerometer and gyroscope on their own, or any radio-frequency based technology or any other technology on its own or combined with any other type of sensor, etc., and/or any other technology and/or methodology and variations and/or combinations thereof may also be used for enhanced accuracy, calibration or any other reasons/purposes. In some embodiments, processing of the sensor data may enable the determination/recognition of certain motion/gait characteristics and/or activity; by way of example without limitation, processing of accelerometer data through the wavelet transform (further details are provided with the description of FIG. 6) or any other methodology and/or combinations thereof may enable the determination of power, energy, frequency components, any kinematic parameter (e.g. user's velocity), peaks distribution over time, step frequency, step length, patterns, any statistics, etc., combinations thereof, or any other type of characteristic/information or any other data or parameter/metric that may or not be in any way related with any characteristic/activity/information, etc., and any or all of those data, metrics, parameters, and/or characteristics, etc. may be leveraged in any fashion to determine/recognize activity or any other information. In other embodiments, any other configuration, methodology, modification and/or combinations thereof may be employed; by way of example without limitation, some embodiments may use any type of technique/methodology (e.g. any type of machine learning technique with training data gathered in any fashion) to recognize activity or other information independently of any other motion characteristic (which may also be determined with any methodology independently, in parallel, in combination, or in any other way regarding recognition of activity or other information), while other embodiments may employ any other methodology, tools, resources, techniques and/or mixtures and/or variations, modifications and/or combinations thereof.

In some embodiments, the gait/motion parameters or characteristics that may be determined/calculated/estimated/inferred include, by way of example without limitation, speed, stride length, cadence, total distance, pace, gait efficiency, energy, power, changes in acceleration, speed variability, strike time, steps, and any combination thereof. In some embodiments, any number of gait/motion parameters and/or any other information may be leveraged to determine additional gait/motion parameters in any way; by way of example without limitation, physics principles may be used to determine distance (e.g. stride length) from velocity, and other parameters or characteristics that may be obtained in this or other fashion include energy consumption, different types of costs, etc. In some embodiments, any variations of any said characteristics or parameters and/or combinations thereof may also be determined in any fashion, and any user's characteristic such as height, weight, gender, age, etc. may also be used to help in the determination of the motion or gait parameters.

Some embodiments may test if the user is performing any type of gait activity, leveraging any of the characteristics/data/methodologies herein mentioned, or through any other methodology; in some embodiments, the type of user's movement that the system tries to recognize may include any activity that may be classified as human gait, in other words, any gait activity, including, by way of example without limitation, any type of walking, jogging, running, sprinting, ascending or descending stairs, exercising on any apparatus such as stationary elliptical trainer or bicycle, and any variation and/or combination thereof regardless of forward/backward direction, flat/inclined surface, type of environment, etc. In some embodiments, any gesture or movement different from walking, jogging or running may not be considered as a gait activity. In other embodiments, the user's movement to be recognized by the system may include any type of movement and/or activity. By way of example without limitation, a particular embodiment may consider walking, jogging, or running as gait activity. Any other variation and/or combination may also be possible.

Any or all of the user's determined characteristics may be leveraged to control any aspect, feature, condition, property or any other attribute of any process, function, procedure, program, application, environment or any other entity or element and/or combinations thereof, in any way.

In some embodiments, irregularities in the user's gait may also be detected through the comparison of any gait characteristic with known regular values stored anywhere in any fashion.

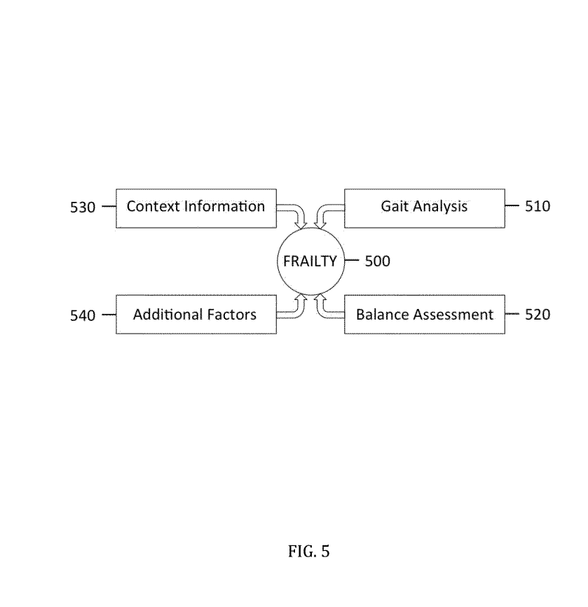

FIG. 5 displays a schematic frailty assessment model according to one embodiment. In particular, gait analysis (510), balance assessment (520), contextual information (530) and additional factors (540) are considered in order to tackle the multifactorial nature of frailty (500) and/or conditions associated with frailty such as falling. Logistic regression may be used for modeling in some embodiments. Other embodiments may use any other approaches, variations, modifications and/or combinations. Regarding gait analysis (510), a plurality of factors may be considered in some embodiments, including by way of example without limitation, gait velocity, step length, cadence, etc. In some embodiments, all the processing to determine said factors may be performed internally within the mobile or wearable device leveraging the sensors embedded within the device, and the information may be presented to the user in real time on the device screen; in other embodiments, distributed or other types of processing, sensing and/or presentation of information may be performed, including any variations, modifications and/or combinations thereof. Some embodiments may also search for irregular gait patterns. Additional information on gait analysis will be included in subsequent paragraphs.

In some embodiments, balance (520) can be assessed measuring variations in the subject's trunk's angles in three dimensions, leveraging the mobile or wearable device's embedded sensors, which may comprise by way of example without limitation, accelerometer, magnetometer and/or gyroscope. In some embodiments, the angle variations can be measured in two dimensions or in one dimension depending on a plurality of criteria and circumstances, including by way of example without limitation, hardware availability, quality of the available hardware, positioning of the device, etc. By way of example without limitation, in some embodiments an accelerometer may be used to estimate tilt angles leveraging the gravity vector, while the magnetometer may provide heading, and the gyroscope may provide angular velocity, and its integration may deliver angles. It is worth noting that the gyroscope may suffer from bias that needs to be considered. In some embodiments, angle(s) variations can be measured in different parts or positions of the subject, including by way of example without limitation, head, shoulders, upper body, sternum, chest, waist level, side, center of mass, pocket, leg, or any other part or position, depending on a plurality of criteria and circumstances, including by way of example without limitation, type of device, characteristics of the device, physical qualities of the subject, personal preference, type of clothes of the subject, etc. In some embodiments, the sensors leveraged to assess angle(s) may comprise accelerometers, magnetometers and gyroscopes, which may be embedded in a single mobile or wearable device, while in other embodiments, the sensors leveraged may comprise a single accelerometer, or a single tri-axial accelerometer, or an accelerometer and a gyroscope, or an accelerometer and a magnetometer, or any other combinations thereof, and any or all of the sensors may be embedded in a single mobile or wearable device. In other embodiments any or all of the sensors may be distributed in any fashion in different devices. In other embodiments, any combinations of any of the possibilities may be employed. In other embodiments, any approaches, methodologies, technologies, sensors, devices, etc. and/or variations, and/or modifications and/or combinations thereof may be used to assess balance.

In some embodiments, common balance tests may be carried out to assess balance through the measurement of variations of angles with a mobile or wearable device. By way of example without limitation, the tests included in the common Balance Error Scoring System may be performed by the subject while carrying or holding the mobile or wearable device in any of the previously referred parts or positions of the subject. In some embodiments any type of test and/or modifications and/or variations and/or combinations thereof may be used. In some embodiments, a variety of stands may be used, including by way of example without limitation, bilateral, unilateral, tandem, with eyes closed, with eyes open, on firm surface, on a foam pad, with any length of time, etc. including any variations, modifications and/or combinations thereof.

In some embodiments leveraging accelerometer, magnetometer and gyroscope to measure angles, a Kalman filter may be used to integrate noisy signals. In other embodiments, any sensor fusion technique or any methodology, technology or technique may be used. In some embodiments leveraging Kalman filtering, wavelet de-noising may be used to pre-process the sensors signals. In some embodiments, all the processing may be done internally in the mobile or wearable device without any external requirements. In other embodiments, any signal processing technique in any combination with any sensor fusion technique or any other methodology, technique or technology may be used. In some embodiments, any variations, modifications and/or combinations are also possible.

In some embodiments, due to a variety of reasons (including by way of example without limitation: the mobile or wearable device may not be equipped with gyroscope, and/or electromagnetic interferences due to normal operations in the mobile or wearable device may severely jeopardize the accuracy of the magnetometer) the balance assessment algorithms may need to adapt to focus on measurements from the accelerometer on its own, leveraging anterior-posterior and medio-lateral coordinates of the Center of Pressure, or by means of any other approaches. In some embodiments, any approaches, techniques, technologies, methodologies and/or variations, modifications, and/or combinations thereof may also be possible.

In some embodiments, contextual information (530), both location and activity information may be determined by the mobile or wearable device, and leveraged to model frailty. In some embodiments, location information may be included for both indoors and outdoors, and all the processing required may be performed internally within the device leveraging embedded sensors; in other embodiments, any aspect of location determination, including processing, may be distributed in any fashion; additional information on localization will be provided in subsequent paragraphs. Some embodiments may also determine user activity information leveraging sensors data; by way of example without limitation, some embodiments may leverage the sensors embedded in the mobile or wearable device to recognize activity, and measure activity levels (e.g. in terms of calories burned or through any other indication); other embodiments may also leverage sensors distributed in other devices in any fashion; some embodiments may carry out all the processing to determine activity information internally within the device; in other embodiments, any aspect of activity information determination, including processing, may be distributed in any fashion. In other embodiments, any variations, modifications and/or combinations of any aspect may also be used.

Some embodiments may use additional factors (540) to model frailty. By way of example without limitation, some embodiments may leverage digitized questionnaires asking for fall history, medication used, chronic diseases, risks related to daily activities, alcohol use, footware used, age, gender, fear of fall, socioeconomic risk factors, environmental risk factors or any other type of information that may help to develop an accurate frailty or fall model in combination with gait analysis, balance assessment, and contextual information. Some embodiments may integrate the referred digitized questionnaires within the mobile or wearable device without the need of external requirements. Some embodiments may leverage the multimedia capabilities of the mobile or wearable device to carry out said questionnaires. Some embodiments may leverage internal sensors of the device or other sensors or other devices or other sources to infer/determine any of the referred or any other information in any fashion. Other embodiments may leverage external resources, devices, capabilities or any other elements for said purposes. Some embodiments may use any variations, modifications and/or combinations of any of the aspects mentioned.

In some embodiments, frailty or any other related information (e.g. fall risk) may be modeled using logistic regression and leveraging the previous factors. In some embodiments, any methodologies, techniques, approaches, and/or variations, and/or modifications and/or combinations thereof may be used for modeling purposes. In some embodiments, a comprehensive application for the mobile or wearable device may include any or all of the mentioned aspects, and additionally include, by way of example without limitation, instructions (in any format including multimedia) on how to perform certain tests to better evaluate frailty, and/or instructions (again in any format) on how to perform certain exercises that may improve the user's well-being, and/or any other type of instructions, feedback or any other information. Other embodiments may consider any variations, modifications and/or combinations of any of the aspects mentioned.

FIG. 6 illustrates a flow diagram of one embodiment with possible basic steps of a method for providing a user's dynamics information. The available sensors in the device are recognized in (610). Some embodiments may employ adaptable algorithms to be able to work with different types of devices (which may have, by way of example, and not limitation, different operating systems, different hardware features, different types of sensors, etc.). In some embodiments, the user's mobile device may have multiple sensors and sensor fusion techniques may be applied to enhance the solution. In other embodiments, the user's device may have very basic functionality and be equipped with a single accelerometer, and the algorithm will adapt to those devices to provide adequate results.

For the purpose of obtaining the dynamics of the user through the processing of sensor(s) signal(s), some embodiments may select an appropriate sampling frequency, which optimizes performance and attempts to minimize power consumption. In some embodiments, it may not be possible to set a desired sampling frequency (620). By way of example, and not limitation, some operating systems may allow the selection of predefined sampling frequency levels, which may work as indicators of the final sampling frequencies, but there is no guarantee of obtaining a specific frequency value. In fact, the final sampling frequency values may also be device and hardware specific. In conclusion, the algorithm in some embodiments will need to adapt to the available sampling frequencies in each particular device. In this sense, the sampling frequency may be selected (630) taking into account two criteria: first, performance optimization; second, power consumption minimization. In fact, optimum performance may depend on the sampling frequency among other factors. In some embodiments, the quality of the results obtained through the application of the wavelet transform to process the sensor(s) (e.g. accelerometer) signal(s) may depend on the sampling frequency. Once the desired or available sampling frequency has been selected, that frequency is set in the device (640). Some embodiments may use single axis sensor information to be processed (by way of example and not limitation, acceleration in x-axis, acceleration in y-axis, acceleration in z-axis). Some embodiments may use the signal vector module to be processed (by way of example and not limitation, the signal vector module of a tri-axial accelerometer). Some embodiments may use different configurations and/or combinations of sensors signals (including but not limited to sensor fusion information) to be processed. It must be noted that in some embodiments, the set frequency may still vary depending on a variety of factors, including but not limited to, device-specific behavior. Consequently, in some embodiments, a frequency resetting procedure may be necessary to maintain desired performance. Some embodiments may use dynamic selection of sampling frequency; by way of example and not limitation, when periods of inactivity are detected, the sampling frequency may be reduced in order to minimize power consumption, and once some activity is detected again, the sampling frequency may be increased again to deliver desired performance.

In some embodiments, the selection of the transformation parameters to process the sensor(s) signal(s) may take place after the sampling frequency is set (650). In some embodiments, the wavelet transform may be applied for processing sensor(s) signal(s). In other embodiments, other transformations may be applied, including but not limited to, short-time Fourier transform, other techniques leveraging Fourier analysis, application of filter banks, etc. In other embodiments different combinations of techniques, methodologies and transformations including wavelets may be used. In some embodiments, the parameters of each transformation, which by way of example and not limitation, may comprise levels of decomposition, mother wavelet, processing time window parameters, etc. may be set appropriately/dynamically to optimize performance and minimize computation burden.

In some embodiments, the appropriate transformation coefficients may be obtained (660) and be leveraged in subsequent processes in combination with other parameters and metrics (670). In some embodiments, the application of metrics with the previously obtained information results in excellent correlations with the velocity of the user, and the activity of the user (e.g. walking, running, jumping, etc.), leading to a characterization of the user dynamics (680). In some embodiments, by way of example and not limitation, weighted (e.g. by levels, number of coefficients, etc.) energies of wavelet transform coefficients may provide an excellent indicator to directly choose the appropriate coefficients from which to obtain a reconstructed wave whose positive-to-negative transitions will mark each step of the user. For instance, the summation of the square of the wavelet transform detail coefficients, divided by the product of the number of detail coefficients at each decomposition level with the total number of decomposition levels plus one minus the actual decomposition level, provides a metric to classify the levels of decomposition; choosing the decomposition level with the highest value of said metric, and applying a reconstruction with its detail coefficients, delivers a wave whose positive-to-negative transitions will mark each step of the user. In some embodiments, useful metrics may comprise the summations of the square of transformation coefficients, these summations scaled by some factor (including but not limited to the number of coefficients, the number of levels of decomposition, a constant, etc.), or any other type of combinations. In some embodiments, the summations of weighted energies of transformation coefficients adequately scaled by some factor (including but not limited to level of decomposition) may provide an excellent correlation with the kinetic energy of the user. For instance, calling weighted energies for each decomposition level to the summation of the square of the wavelet transform detail coefficients, divided by the product of the number of detail coefficients at each decomposition level with the total number of decomposition levels plus one minus the actual decomposition level, and applying the square root to the summation of said weighted energies for each decomposition level being divided by the actual level, delivers an estimation of the velocity. In some embodiments, some of the coefficients may be avoided for the calculation of metrics, and appropriate combinations of summations of weighted energies may be leveraged to compute information comprising velocity. In some embodiments, criteria to avoid transformation coefficients in the calculation of metrics may comprise: selection of a threshold, frequency content, etc. Some embodiments may leverage statistics (including but not limited to, range, mean, skewness, standard deviation, etc.) of the energies of transformation coefficients, or any other features or combinations thereof to be combined with the previously mentioned computed kinematic information and obtain user dynamics information comprising activity. By way of example and not limitation, some embodiments may leverage as metrics the summations of descriptive statistics (or combinations of them) of energies of transformation coefficients of predetermined levels (choice criteria may comprise threshold, frequency content, etc.), in combination with other summations of descriptive statistics (or combinations of them) of energies of transformation coefficients of predetermined levels (choice criteria may again comprise threshold, frequency content, etc.), in combination with velocity information.

Some embodiments may leverage the previously mentioned information about the user's steps in combination with other metrics to enhance user's dynamics information, comprising velocity and activity. Some embodiments may leverage the obtained information on user's steps in combination with the information on user's dynamics to determine stride length. Some embodiments may leverage the information on user's dynamics to compute distance. Some embodiments may enhance distance through the combination of user's dynamics information with localization information. Some embodiments may use different techniques, principles and/or methodologies to obtain all the previous information and metrics, including but not limited to machine learning. In some embodiments, all the computation, processing, information presentation, and other steps may be carried out within a single mobile device without the need of external resources. In some embodiments, the computation or some other step or combinations of steps may be performed external to the mobile device, or with the assistance of some external element, such as external sensor, server, database or any other element. In some embodiments, software may be stored on the mobile or wearable device, for instance, in its memory for execution by its processor or processors. Some embodiments may store data structures and code on computer readable storage medium, which by way of example, and not limitation, may comprise field-programmable gate arrays, application-specific integrated circuits, magnetic and/or optical storage devices, etc.

In some embodiments, the sensor portion of the device or the device itself or any other device containing a sensor and with the capability to communicate in any fashion with the user's device, or any other type of device or accessory may be positioned or attached to any part of the user, including by way of example without limitation, the wrist, arm, hand, face, head, waist, chest, pocket, hat, shoe, any type of clothing, accessories and any combinations thereof and in any way. In some embodiments, the system may be trained to recognize and/or learn activity, motion type, attachment position of the device, movement characteristic, etc. In some embodiments, analysis of acceleration signature may help determine activity, motion type, attachment position of the device, movement/gait characteristic, etc. By way of example without limitation, the acceleration signal may be processed to identify maximums, minimums, mean, standard deviation, frequency components, period, orientation, distribution of peaks, patterns, etc. and/or combinations thereof in order to help determine activity, motion type, attachment position of the device, movement/gait characteristic, etc. In some embodiments, Fourier analysis, any kind of filtering, peak counting, determination of frequency components leveraging the wavelet transform or any other method and combinations thereof may also be utilized to determine user's gait activity, characteristics, etc. In some embodiments, any type of prompt to the user may also be leveraged to request information about his/her activity, motion type, attachment position of the device, movement/gait characteristic, etc. In some embodiments, activity, motion type, attachment position, movement/gait characteristic, etc. may be determined through correlation of any type of sensor values or any type of parameter or metric generated with them, based on any type of model that has been calibrated in any fashion for a particular activity, motion type, attachment position, movement characteristic, etc. In some embodiments, any other sources, means, methods and/or configurations may be leveraged to determine activity, motion type, attachment position, movement/gait characteristic, etc., including by way of example without limitation, the use of sensors and/or signals obtained independently of the sensed acceleration (e.g. GPS), the use of statistics and/or any other empirical information, algorithms, databases or other information stored anywhere and in any fashion, combinations thereof, etc. In some embodiments, the referred methods, configurations, systems, etc. may be modified, updated and/or calibrated in any way, periodically or continuously over any time interval.

Some embodiments may include any external sources to obtain any parameter or information about movement, environment, context, etc. including by way of example without limitation, speed and/or distance monitors, any number of portable electronic devices (e.g. GPS receivers, any kind of computing and/or communications device, etc.), databases and/or networks. In some embodiments, other types of inputs may also be utilized, including by way of example without limitation, buttons, keys, keyboards, keypads, touchpads, joysticks, etc., which may be used in any fashion. Any type of satellite based navigation systems, cellular communications networks and other systems/networks may also be used to obtain speed in some embodiments (and/or provide feedback to help correct errors) under certain conditions.

In some embodiments, additional inputs may include traces from touch-sensitive screens, button presses, gesture recognition, voice commands, switches, and/or any other type of technological, physical or any nature means that allow the user to interact, and combinations thereof. In some embodiments, any type of method may be employed to distinguish between different types of gestures, swings, twists, etc. that the user makes while he/she performs a pedestrian activity (e.g. walk, jog, run, etc.); by way of example without limitation, frequency analysis, filtering, acceleration thresholding, analysis of projection of gravity vector, feedback from other sensors, or any other technique/method and combinations thereof may be employed.

In some embodiments, the acceleration sensor may be an electrostatic or capacitance-coupling type, or any other technology (e.g. piezoelectric or piezoresistance type) now existing or later developed, and may be configured to deliver three-axis, two-axis, or one-axis acceleration. In some embodiments, in addition to accelerometers, any other type of technologies and/or sensors such as gyroscopes, magnetometers, pressure sensors, cameras, GPS, etc. may be used in any way to enhance accuracy or for any other purposes. In some embodiments, the user may have any number of any type of sensors, sensor units, devices, or accessories located anywhere in any fashion to determine the characteristics of his/her movement and/or for control or any other purposes.

In some embodiments, any processing, detection, recognition, or any other actions or operations may be performed regardless of the mode, state or any other condition of the device, application or any other entity, process or element. In other embodiments, any number of conditions and/or criteria of any type must be satisfied before proceeding with any of said actions or operations.

Any of the embodiments herein described may be implemented in numerous ways, including as a method, an apparatus, a device, a system, a computer readable medium, etc., and also be applicable in any environment, application, condition, etc. regardless of number of users, physical proximity, communication means, device, or any other factor.

Other configurations are also possible. By way of example, and not limitation, in some embodiments, all or part of the processes may be performed by chip-level systems, third-party applications, operating system kernel, firmware, or any other combination of hardware and/or software. In some embodiments, the software may be delivered in a variety of forms, including but not limited to, as stand-alone application, as library, as application programming interface, etc. In general, the functions of particular embodiments may be achieved by any means as is known in the art. Some embodiments may use distributed, networked sensors and/or systems, components, servers, databases, and/or circuits, and/or any combination of additional hardware and/or software and/or processing techniques and methodologies. Some embodiments may use any other type of sensor and/or system.

In some embodiments, sensors may be any of several types including, by way of example, and not limitation, any type of device, transducer or any other type of apparatus which may measure some quantity; in some embodiments, sensors may be implemented in any size, with any type of technique and technology, including but not limited to electronic, microelectronic, nanoelectronic, etc. By way of example, and not limitation, sensors may comprise any type of accelerometer, magnetometer, gyroscope, pressure sensor, proximity sensor, etc. and any other type of device sensitive to radio-frequency, sound, ultrasound, light, etc. including but not limited to, GPS antennas and/or their sensitive elements, WiFi antennas and/or their sensitive elements, and any other type of radio-frequency technology antennas and/or their sensitive elements. In some embodiments, sensors are integrated within the mobile or wearable device. In some embodiments, sensors or other mobile or wearable devices may be distributed outside the main mobile or wearable device, and they may communicate with the main mobile or wearable device by any means. Communication or transfer of data may be wired, wireless, or by any other means. In some embodiments, the user or other entity may rearrange characteristics of the components, or other features or elements of the system and the system may automatically adjust to new settings or arrangements.

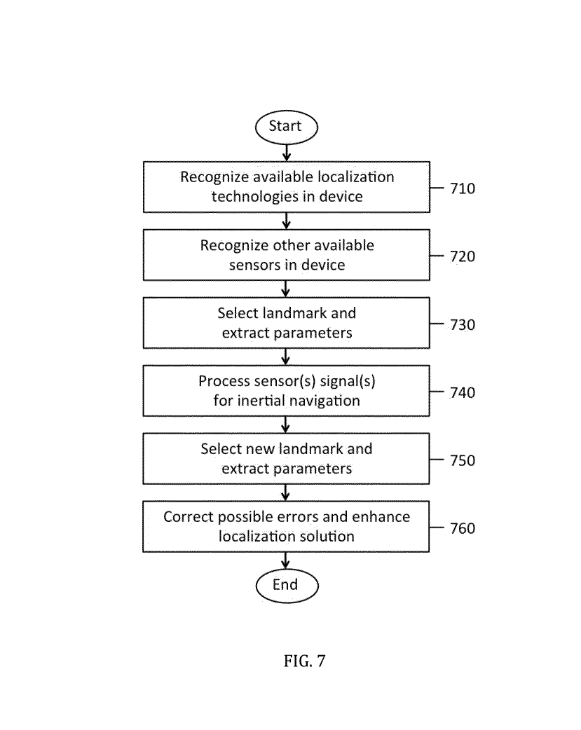

In some embodiments, a method for enhancing a user's dynamics and localization information may be used as shown in FIG. 7, which illustrates a flow diagram of possible basic steps. The available localization technologies are recognized in (710). By way of example and not limitation, localization technologies or methodologies may include satellite-based systems such as GPS, radio-frequency fingerprinting based techniques, and others based on various techniques, principles and/or technologies, including their combinations through a variety of methodologies such as Kalman filtering, particle filtering, etc. Regarding the radio-frequency fingerprinting based techniques, several technologies may be employed, including but not limited to, WiFi, cellular, Bluetooth, Zigbee, digital television, etc. In some embodiments, the use of satellite-based localization technologies may be avoided because the user may be located within buildings, urban canyons, or other environments in which the performance of these technologies is degraded. Even in those outdoor environments where the device may receive good quality signal from the satellites, these satellite-based systems may be avoided due to their high power consumption. In some embodiments, other localization techniques, technologies and methodologies may be used, including but not limited to, Near Field Communications, Ultra Wide Band, acoustic, ultrasound, any type of radio-frequency, etc. The available sensors in the device are recognized in (720). In some embodiments, these sensors may include accelerometer, magnetometer, gyroscope, pressure sensor, and others. In some embodiments, the device may include very basic functionality and the algorithm may need to adapt and perform efficiently with a single accelerometer. In other embodiments, the sensors in the device may include more than a single accelerometer, and sensor fusion techniques may be used. In other embodiments, other configurations of sensors may be possible.

In some embodiments, recognizable places may be set as landmarks from which to extract very precise features regarding their location and general context (730). By way of example and not limitation, Radio Frequency Identification, Bluetooth, Zigbee and/or other technologies and/or combinations of them may be leveraged using a variety of techniques to identify landmarks with a very high resolution. Leveraging the information on the user's dynamics, some embodiments may obtain accurate inertial navigation information (740). In some embodiments with basic functionality where the device may not be equipped with gyroscope and/or magnetometer, a variety of mechanisms to identify straight-line trajectories may be leveraged to adapt the inertial navigation solution. When a new identifiable landmark is reached, location and general context features are extracted (750). By way of example and not limitation, some embodiments may use GPS outdoors, or radio beacons indoors detected as peaks in signal strength within a radio-fingerprinting localization system, to identify landmarks. In other embodiments, the use of other types of beacons or landmarks, derived from a variety of technologies, that may use a variety of principles to obtain the required information, is also possible. This information may be leveraged using a variety of possible techniques and methodologies to correct possible errors on the user's dynamics and enhance the localization solution (760). Some embodiments may use manual calibration by the user introducing required calibration parameters in ways he/she may choose from a variety of techniques, technologies and methodologies. Other embodiments may use automatic calibration. In some embodiments, the calibration may be successfully applied to enhance both the information on localization and the user's dynamics and contextual information.

Some embodiments may use all the available information to identify the position (and transitions between positions) of the mobile device within the user's body; by way of example and not limitation, the position information may comprise: held in front in reading position, held in hand while walking, held in pocket while walking, etc. Some embodiments may use external elements comprising user's input to identify positions; in other embodiments, positions will be recognized internally by the mobile device leveraging sensors information.

Some embodiments may use any type of smartphones, mobile devices, wearable devices and/or sensors, or any other types of devices or combinations of them, including but not limited to, personal digital assistants, personal navigation systems, portable electronic devices, tablets, laptops, computers, and their peripheral devices. In some embodiments, the definition of mobile device may comprise any type of mobile phone, smartphone, wearable device and/or sensor, or any other types of portable device or wearable or combinations of them.

Some embodiments may use combinations of strategies and techniques, including, by way of example, and not limitation, machine learning techniques, probabilistic models, sensor fusion techniques, extraction of statistics, employment of filter banks, application of dimensionality reduction techniques, a variety of approaches for classification, etc. Details are omitted to improve the clarity of the description. In addition, some embodiments may use a variety of programming languages and methodologies in combination with varied hardware configurations and execution strategies.

Some embodiments may leverage context information and provide supplemental information, which may be obtained through any means and sources, including but not limited to, social networks. Particular embodiments may also be used for targeted advertising or targeted information based on context, enable shopping of any type of product or service which may or may not be related to the contextual information, etc.

In some embodiments, various applications may use the obtained information as a trigger for activation. Alternatively, a user may be able to set preferences for different applications depending on the obtained information. By way of example, and not limitation, a user may set the font size and other features of the content (also obtainable through internet or any other means) in his/her mobile device display according to his/her dynamics to improve the reading experience. By way of example, and not limitation, the user may or may not have ear-speakers or head-phones or any other appropriate hardware connected to his/her device and he/she may opt for triggering an out-loud-reader or other type of application to read-out-loud or in some other way adapt the presentation of the content in the device display when his/her dynamic information stays within some preselected threshold levels. By way of example, and not limitation, application(s) and/or service(s) may request, trigger or in some way enable advertising from a commercial ad server or any other type of server or entity using either velocity information, user dynamics, key words, or other criteria as advertising keys. In some embodiments, the user's velocity and other information, including advertisements, may be presented on the mobile and/or wearable device for consideration by the user. Depending on preferences and personal privacy policies, information may be presented to desired friends or other people.

Applications of some embodiments may comprise monitoring a variety of information of people in a variety of circumstances or contexts, including but not limited to, health-care, army, sports, etc. Some embodiments may perform the monitoring in a remote way and/or extend the monitoring to animals, robots, machines, etc. In some embodiments, services may be provided through subscription. Some embodiments may be applied for the estimation of calories consumption, or for the monitoring, diagnosis or other procedures related to diseases such as Parkinson's or other neurodegenerative diseases. Some embodiments may be applied for the identification and/or treatment of disorders, such as gait disorders, associated with a wide variety of conditions, including but not limited to neurologic and orthopedic conditions. Some embodiments may obtain a wide variety of user's information, including but not limited to velocity, activity, stride length, cadence, step count, gait patterns, contextual information, balance, frailty, etc. in real time. Some embodiments may apply the information to help in the prevention of falls, accidents or any other undesirable events. Applications of some embodiments may also include contextual interactions, interactive games, augmented reality, and other types of services.

In some embodiments, the obtained information may be used for social networking applications, such as finding and/or establishing communication and/or sharing information with friends and/or other people and/or groups of people whose contextual information might or might not in some way be related. By way of example, and not limitation, in some embodiments, users may be able to share and see the real-time and/or historical contextual information of their friends, edit contextual information on maps, etc. In some embodiments, the observation of two or more mobile and/or wearable devices following similar contextual patterns, may lead to infer a friendship.

Some embodiments may also be applied to infer information from a wide range of biological or other types of sensors/signals, either from humans, animals, mechanical entities such as robots or other machines, etc. Other embodiments may also be applied to monitor and optimize a variety of processes, including but not limited to, industrial and managerial processes. Other embodiments may also have many more applications.

Although the foregoing text sets forth a detailed description of numerous different embodiments of the invention, it should be understood that the scope of the invention is defined by the words of the claims set forth at the end of this patent. The detailed description is to be construed as exemplary only and does not describe every possibly embodiment of the invention because describing every possible embodiment would be impractical, if not impossible. Numerous alternative embodiments could be implemented, using either current technology or technology developed after the filing date of this patent, which would still fall within the scope of the claims defining the invention.

Thus, many modifications and variations may be made in the techniques and structures described and illustrated herein without departing from the spirit and scope of the present invention. Accordingly, it should be understood that the methods and apparatus described herein are illustrative only and are not limiting upon the scope of the invention.

1. A method for real time monitoring of a mobile and/or wearable device user, the method comprising:
obtaining sensors data, where sensors comprise accelerometer;
obtaining the squares of wavelet transformation coefficients of accelerometer data;
weighting the squares of wavelet transformation coefficients; obtaining the summation of said weighted squares, and leverage said summation to estimate the velocity of the user;

2. The method of claim 1, further comprising:
obtaining an indication of the user's balance using the mobile or wearable device sensors data.

3. The method of claim 2, further comprising:
obtaining location information leveraging sensors data;
combining user's velocity with location information for enhanced localization and calibration.

4. The method of claim 3, further comprising:
leveraging weighted energies of the accelerometer wavelet transformation coefficients to choose the coefficients from which to obtain a reconstructed wave from where each step of the user is clearly identified;
combining step time information with velocity estimation to estimate step length.

5. The method of claim 2, wherein obtaining the balance indication comprises wavelet de-noising and Kalman filtering with the sensors data.

6. The method of claim 1, further comprising: leveraging said velocity to estimate calories burned per time unit.

7. The method of claim 6, further comprising: displaying in real time in the device screen the determined calories burned per time unit.

8. A method for real time monitoring of a mobile or wearable device user, the method comprising:
obtaining sensors data;
obtaining the wavelet transformation coefficients of sensors data;
obtaining an indication of the user's balance using wavelet de-noising on the sensors data and Kalman filtering with said de-noised data.

9. A system comprising:
a processor;
a non-transitory processor-readable medium including one or more instructions which, when executed by the processor, causes the processor to monitor a mobile and/or wearable device user in real time by:
obtaining sensors data, where sensors comprise accelerometer;
obtaining the squares of wavelet transformation coefficients of accelerometer data;
weighting the squares of wavelet transformation coefficients; obtaining the summation of said weighted squares, and leverage said summation to estimate the velocity of the user.

10. The system of claim 9, wherein the monitoring a mobile and/or wearable device user in real time, further comprises: obtaining an indication of the user's balance using the mobile or wearable device sensors data.

11. The system of claim 10, wherein the monitoring a mobile and/or wearable device user in real time, further comprises:
obtaining location information leveraging sensors data;
combining user's velocity with location information for enhanced localization and calibration.

12. The system of claim 11, wherein the monitoring a mobile and/or wearable device user in real time, further comprises:
leveraging weighted energies of the accelerometer wavelet transformation coefficients to choose the coefficients from which to obtain a reconstructed wave from where each step of the user is clearly identified;
combining step time information with velocity estimation to estimate step length.

13. The system of claim 10, wherein obtaining the balance indication comprises wavelet de-noising and Kalman filtering with the sensors data.

14. The system of claim 9, wherein the monitoring a mobile and/or wearable device user in real time, further comprises: leveraging said velocity to estimate calories burned per time unit.

15. The system of claim 14, wherein the monitoring a mobile and/or wearable device user in real time, further comprises: displaying in real time in the device screen the determined calories burned per time unit.
